(19) United States
(12) Patent Application Publication
(10) Pub. No.: US 2006/0247787 A1
(43) Pub. Date: Nov. 2, 2006

(54) Method and system for toe arthroplasty

(76) Inventors: Mark A. Rydell, Golden Valley, MN (US); Scott McGarvey, Edina, MN (US); Jeffrey C. Felt, Greenwood, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/562,651

(22) PCT Filed: Jun. 25, 2004
(86) PCT No.: PCT/US04/20457

§ 371 Date: Jun. 30, 2006

Related U.S. Application Data
(60) Provisional application No. 60/483,499, filed on Jun. 27, 2003.

(51) Int. Cl. A61F 2/42

(52) U.S. Cl. 623/21.11

(57) ABSTRACT

A system for the creation or modification of an orthopedic joint within a mammalian body is disclosed. The system includes an implant (122, 132, 232, 332, 432) that provides a major surface adapted to be positioned against a metatarsal bone and an end (136, 236, 336, 436) adapted to be retained within a phalange.

### TECHNICAL FIELD

In one aspect, this invention relates to biomaterials for implantation and use within the body. In yet another aspect, this invention further relates to the field of orthopedic implants and prostheses, and more particularly, for implantable materials for use in orthopedic joints, such as the great toe joint.

### BACKGROUND OF THE INVENTION

Applicant has previously described, inter alia, prosthetic implants formed of biomaterials that can be delivered and finally cured in situ, and/or that can be partially or fully prepared ex vivo, for implantation into the body, e.g., using minimally invasive techniques. See for instance, U.S. Pat. Nos. 5,556,429; 5,795,353; 6,140,452; 6,306,177; and 6,652,587, as well as US Application Publication Nos. US-2002-0156531; US-2002-0127264; US-2002-0183850; and US-2004-0107000, and International applications having Publication Nos. WO 95/30388; WO 98/20939; WO 02/17821; WO 03/053278; WO 03/061522, and WO 2004/006811 (the disclosures of each of which are incorporated herein by reference).

In spite of developments to date, there remains a need for a joint prosthesis system that provides an optimal combination of properties such as ease of preparation and use, and performance within the body, and particularly for use in joints other than the knee.

### SUMMARY OF THE INVENTION

The present invention provides an interpositional arthroplasty system for use in repairing joints located in a foot, such as a metatarsophalangeal joint (MTP). In some embodiments, the system includes an implant designed to be positioned in the first MTP joint (sometimes referred to as the great toe joint). Such an implant is useful for correcting various deformities of a toe and increasing articulation of a joint.

In some embodiments, the implant is generally funnel shaped, optionally including a barbed fixation shank, and can be press fit into a reamed out cavity in the phalanx bone. The load-bearing surface of the implant can be textured (e.g., dimpled) to provide any suitable size and shape, e.g., to match the radius of the metatarsal head. The peripheral edge of the load-bearing surface can include a radius to mitigate dorsal impingement against the metatarsal head.

In some embodiments, the design of the implant is substantially anatomically accurate. The phalanx can be free to move over the metatarsal and can be constrained by ligaments, capsule, and/or soft tissue. In turn, some embodiments of this invention include preservation of the plantar condyles of the first metatarsal for weight bearing articulation with the sesamoids. By reestablishing the physiologic stabilizing structures of the metatarsophalangeal joint, a balanced and efficient functional joint is promoted. The proper size implant can be chosen depending on the size (e.g., diameter) and the desired amount of ligament tension. The implant can articulate against the metatarsal bone, which in some embodiments, can be surgically smoothed to remove roughness while retaining cartilage.

The implant can be made of one or more biomaterials such as polymers, ceramics, and/or metals. In some embodiments, the biomaterial used in the implant has a modulus that is less than that of bone and can provide a low friction shock absorbing structure. Some embodiments of the invention include a polymeric implant that provides a major surface adapted to be positioned against a metatarsal bone and an end adapted to be fixedly retained within a phalange. Other embodiments include a polymeric implant shaped for interpositional arthroplasty with a major surface adapted to be positioned against a metatarsal bone and a second major surface adapted to be retained against a phalange. In such an embodiment, the second major surface may be designed for congruency against the phalange and the first major surface may be adapted for articulation with the metatarsal.

Some embodiments of the system can also include one or more devices in the form of a kit that can be used to provide or perform some or all of the steps of preparing the joint to receive an implant, determining an appropriate implant size for a particular joint, determining an appropriate implant thickness and/or angle, inserting the implant into the joint, and/or securing the implant to a desired extent. One or more of the various components and devices, including optionally one or more implants themselves, can be provided or packaged separately or in varying desired combinations and subcombinations to provide a kit of this invention. Further, the invention also includes a method of repairing a joint located in the foot, such as a MPT.

In preferred embodiments, the invention provides a prosthetic device for implantation into a toe joint space within the body of a mammal, the device comprising a composite or monolith structure fabricated from a biocompatible, biodurable material that is adapted to be inserted into the joint compartment. More preferably, the implanted device is substantially free of anchoring portions that need to be attached to the bone, cartilage, ligaments or other tissue, yet by its design is capable of being used with minimal translation, rotation, or other undesired movement or dislocation within or from the joint space. The stability of the device within the joint space is provided, in whole or in part, by the fixation/congruency of the device to the one or the other, and generally the relatively less mobile, of the two joint members.

### BRIEF DESCRIPTION OF THE DRAWING

FIG. 1 is a side view of a foot including both an implant of this invention and a plurality of bones making up the joint.

FIG. 2(a) is a side view of an implant in accordance with an exemplary embodiment of the present invention.

FIG. 2(b) is an end view of an implant in accordance with an exemplary embodiment of the present invention.

FIG. 3(a) is a side view of an implant in accordance with an additional exemplary embodiment of the present invention.

FIG. 3(b) is an end view of an implant in accordance with an additional exemplary embodiment of the present invention.

FIG. 4(a) is a side view of an implant in accordance with another exemplary embodiment of the present invention.

FIG. 4(b) is an end view of an implant in accordance with another exemplary embodiment of the present invention.

FIG. 5(a) is a side view of an implant in accordance with yet another exemplary embodiment of the present invention.

FIG. 5(b) is a detail view of an implant in accordance with yet another exemplary embodiment of the present invention.

FIG. 6(a) is a elevation view of a device in accordance with an exemplary embodiment of the present invention.

FIG. 6(b) is a plan view of a device in accordance with an exemplary embodiment of the present invention.

FIG. 7(a) is a side plan view of a foot with an implant in accordance with an embodiment of the present invention.

FIG. 7(b) is a top plan view of a foot with an implant in accordance with an embodiment of the present invention.

FIG. 8 is a top plan view of an exemplary embodiment of a kit in accordance with the present invention.

### DETAILED DESCRIPTION

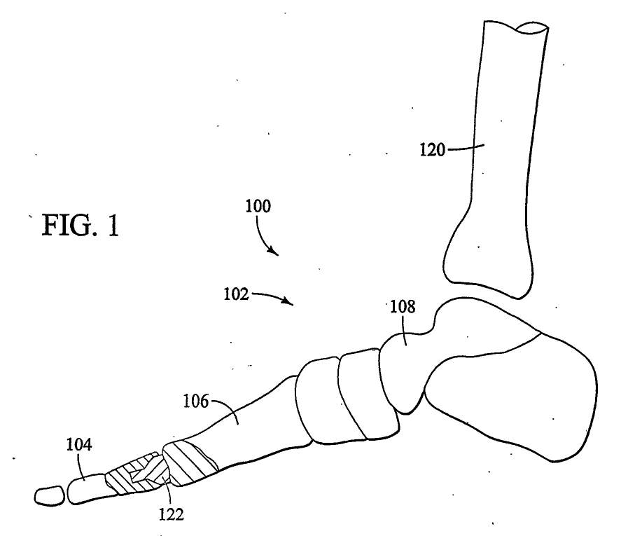

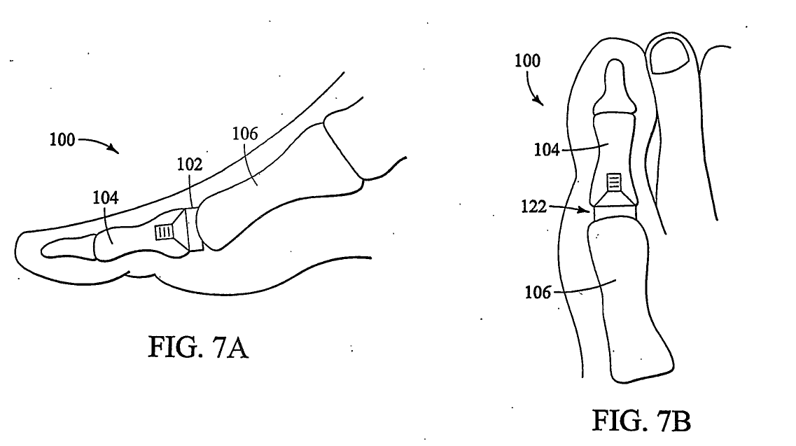

A preferred embodiment will be described with reference to the figures, where FIG. 1 is a side view of a foot 100 including a plurality of bones 102. The bones of foot 100 include a first phalanges 104. A metatarsal bone 106, and a talus 108. A tibia 120 is also shown in FIG. 1. In the embodiment of FIG. 1 an implant 122 is disposed between first phalanges 104 and metatarsal bone 106 proximate the first metatarsophalangeal joint. Additional views of foot 100 and implant 122 are provided in FIGS. 7(a) and (b).

Implant 122 may be provided in a variety of sizes. For example, implant 122 can be provided in diameters from about 5 to about 25 millimeters. Preferably, implants 122 can be provided in diameters of 10, 13, and 16 millimeters. The height of the implant 122 may range from about 0.5 millimeters to about 10 millimeters. Preferably, each diameter of implant 122 is provided in at least heights of 2, 4, and 6 millimeters. In this case, the height of the implant 122 refers to the extent of which it sits above the phalange 104.

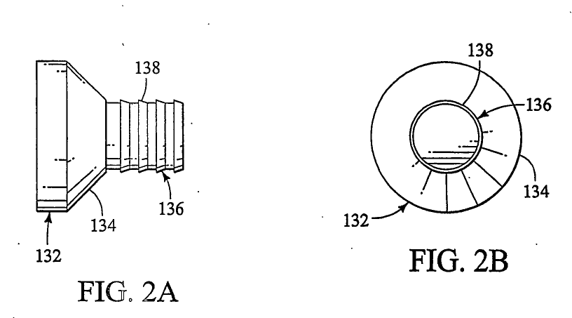

FIGS. 2(a) and (b) include a side view and an end view showing an implant 132 in accordance with an exemplary embodiment of the present invention. Implant 132 comprises a body 134 and, in some embodiments, a shank 136. Suitable structural components, e.g., stabilization means, can be provided to stabilize (e.g., fix) the implant 132 within the joint. Such structures can include the use of barbs, sharp points, splines, diamond patterns or the like. In the embodiment of FIGS. 2(a) and (b), a plurality of barbs 138 are disposed on shank 136 of implant 132. In some advantageous embodiments of the present invention, barbs 138 are shaped and dimensioned so as to reduce the likelihood that implant 132 will migrate out of position. In the embodiment of FIGS. 2(a) and (b), each barb 138 extends substantially entirely around the circumference of shank 136. When this is the case, barbs 138 can provide a seal against fluid moving in and out of a bone cavity receiving shank 136.

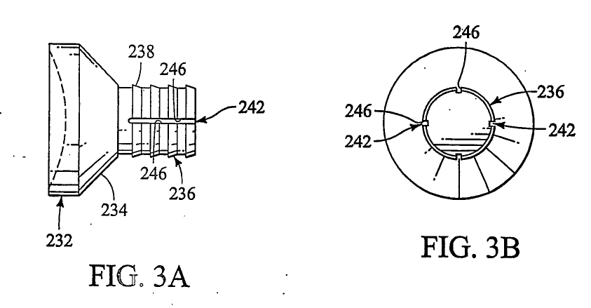

FIGS. 3(a) and (b) include a side view and an end view showing an implant 232 in accordance with an additional exemplary embodiment of the present invention. Implant 232 comprises a body 234 and a shank 236. In FIGS. 3(a) and (b), a plurality of barbs 238 can be seen disposed on shank 236 of implant 232. In the embodiment of FIGS. 3(a) and (b), barbs 238 and shank 236 define a plurality of lengthwise grooves 242. With reference to FIGS. 3(a) and (b), it will be appreciated where each barb 238 is transected by a groove 242, the barb has a plurality of sharp points 246. These sharp points 246 can interact with a bone receiving shank to reduce the likelihood of rotation of implant 232 relative to the bone.

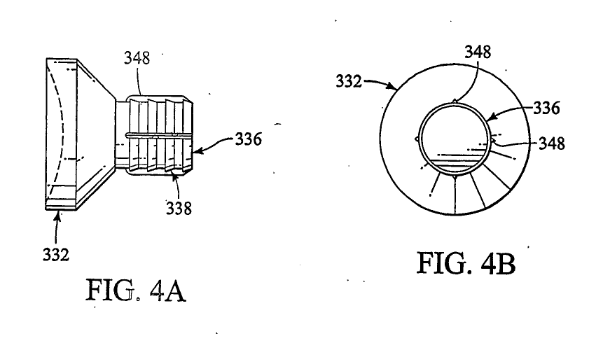

FIGS. 4(a) and (b) include a side view and an end view of an implant 332 in accordance with another exemplary embodiment of the present invention. In the embodiment of FIGS. 4(a) and (b), implant 332 includes a plurality of splines 348 and a plurality of barbs 338 that are disposed on a shank 336. With reference to FIGS. 4(a) and (b), it will be appreciated that each spline 348 extends beyond barbs 338. In the embodiment of FIGS. 4(a) and (b), each spline 348 is generally triangularly shaped in cross section. Splines 348 can advantageously interact with the bone to reduce the likelihood that implant 332 will rotate in situ.

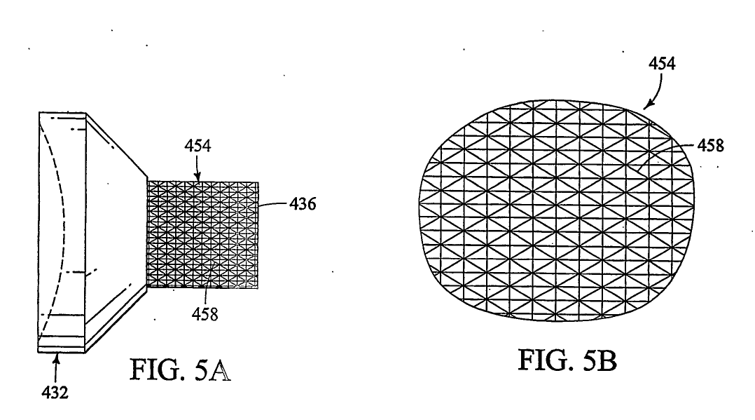

FIGS. 5(a) and (b) include a side view and a detail view of an implant 432 in accordance with yet another exemplary embodiment of the present invention. In the embodiment of FIGS. 5(a) and (b), shank 436 of implant 432 includes a diamond pattern 454. Diamond pattern 454 can act to reduce the likelihood that lateral displacement or rotation of implant 432 will occur in situ. Embodiments of implant 432 are possible in which diamond pattern 454 is combined with one or more barbs. In some applications, these barbs can provide a seal against fluid moving in and out of the bone cavity. Detail view FIG. 5(b) further illustrates diamond pattern 454. With reference to FIG. 5(b), it will be appreciated that diamond pattern 454 comprises a plurality of generally diamond shaped projections 458.

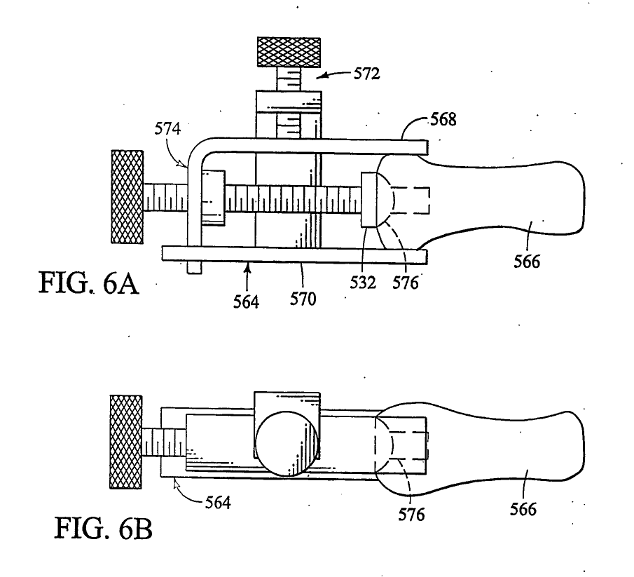

FIG. 6(a) includes a plan view and FIG. 6(b) includes an elevation view of a device 564 in accordance with an exemplary embodiment of the present invention. Device 564 can be used, for example, to press an implant 532 into place in a bone 566. In some methods in accordance with the present invention, an implant can be hammered into place. In some cases, however, hammering an implant into place can be difficult. For example, this may not be easy to do with very small bones like those found in the foot.

In the embodiment of FIGS. 6(a) and (b), bone 566 is disposed between a first jaw 568 and a second jaw 570. Embodiments of the present invention are possible in which first jaw 568 and/or second jaw 570 include sharpened points to pierce into bone 566. In FIGS. 6(a) and (b), a first screw arrangement 572 is shown urging first jaw 568 and second jaw 570 toward one another so as to clamp bone 566. A second screw arrangement 574 can be used to force implant 532 into a hole 576 in bone 566. Embodiments of the present invention are possible in which device 564 is arranged like a pliers with a set of handles and jaws to grab the bone and another lever to press the implant into place.

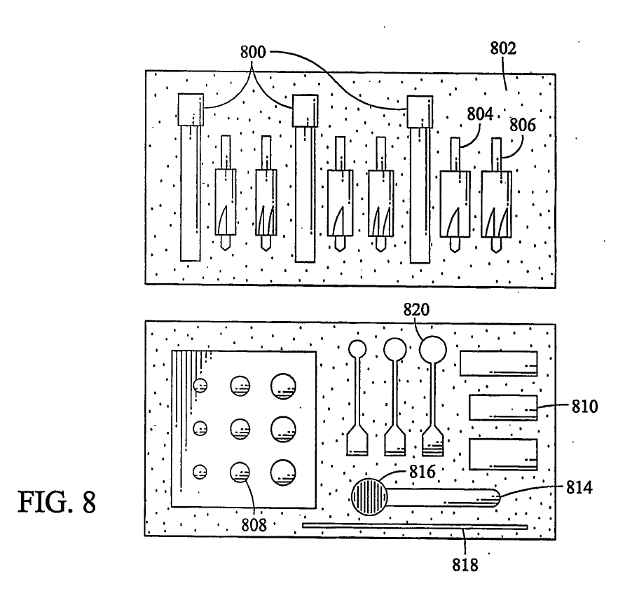

Some embodiments of the system can also include one or more devices in the form of a kit, as shown in FIG. 8, that can be used to provide or perform some or all of the steps of preparing the joint to receive an implant, determining an appropriate implant size for a particular joint, determining an appropriate implant thickness and/or angle, inserting the implant into the joint, and/or securing the implant to a desired extent. One or more of the various components and devices, including optionally one or more implants themselves, can be provided or packaged separately or in varying desired combinations and subcombinations to provide a kit of this invention.

In some embodiments, several implants are included in the kit. For example, implants can be provided in three diameters and three heights to accommodate structural variations of the metatarsophalangeal dimensions. In some embodiments, implants with diameters of 10, 13, and/or 16 mm can be provided. Further, implants with heights of 2, 4, and/or 6 mm can be provided. In this instance, “height” represents the amount that the implant projects above the surface of the phalanx. Choosing from these sizes influence the amount of ligament tension in the joint.

In some embodiments, at least one impactor 800 can be provided, as shown in FIG. 8. Impactor 800 is useful for placing the implant within a joint. In some embodiments, impactor 800 is designed to hold an implant at one end, and to be struck by a mallet at the opposite end to place an implant within a joint. In some embodiments, impactors 800 of several diameters are provided in order to hold implants of different sizes.

In some embodiments, at least one reamer 804 can be provided, as shown in FIG. 8. Reamers 802 are useful for removing bone and natural tissues in the i phalange so that the implant may be placed. Reamers 802 may be powered by a surgical drill. In some embodiments, two reamers for each implant diameter are provided. A first reamer 804 can be used as a pilot reamer for drilling a pilot hole and squaring off the end of the phalanx. A second reamer 806 can be used for creating a tapered area of the cavity.

In some embodiments, at least one depth stop 810 can be provided, as shown in FIG. 8. Depth stop 810 can be slipped over the reamer 802 to prohibited drilling beyond a desired depth. Some embodiments of the depth stop 810 have a shoulder that contacts the bone.

In some embodiments, at least one bone smoother 814 can be provided, as shown in FIG. 8. Bone smother 814 is useful for smoothing and removal of marginal osteophytes from the lateral dorsal, and medial aspects of the metatarsal head that would interfere with the motion of the implant. Smoother 814 can be provided with a textured concave surface 816 that can be used to smooth the metatarsal by positioning in contact with the head and manipulating it back and forth. Smoother 814 can be fenestrated. Such embodiments are useful for simultaneously smoothing a phalange and a metatarsal, as well as for providing for self-cleaning by allowing debris to pass between the superior and inferior sides.

In some embodiments, at least one wire 818 can be provided, as shown in FIG. 8. Wire 818 is useful for guiding the reamers 802 into position. In some embodiments, wire 818 is a 1.6 mm K-wire.

In some embodiments, at least one diameter gauge 820 can be provided, as shown in FIG. 8. Diameter gauges 820 are useful for determining the proper implant diameter for a patient. Diameter gauges 820 can be provided individually as shown in FIG. 8, or a plurality of different sized heads can be provided on a single tool. In some embodiments, the diameter size of the gauge is engraved into the tool.

In some embodiments, at least one trial implant 828 can be provided, as shown in FIG. 8. Trial implants 828 are useful for confirming the proper implant diameter and height before the implant is placed. In some embodiments, trial implants 828 do not have stabilizing means on the fixation shank, so press fitting into the bone is not required.

The tools described above can be constructed of any suitable material. For example, the tools can be constructed of stainless steel, ceramic, and/or polymeric materials. Embodiments constructed at least partially of stainless steel can be relatively more suitable for providing a reusable tool, and embodiments constructed at least partially of a polymer can be relatively more suitable for providing a disposable tool. Further, all of the tools above can be shaped to provide an ergonomic fit for the user. Some embodiments provide a universal tool that is configured to provide an ergonomic fit for both left and right hands and/or can be used both the right and left foot.

In one exemplary method in accordance with the present invention, an incision is made at the base of the great toe and the toe is dis-articulated exposing the end of the first phalanges bone. A wire is drilled into the bone axially to guide the drills. A reamer that fits over the wire is used to create a pilot hole. A second reamer can then used to create a tapered countersunk area for the implant to reside in. The implant can be, for example, hammered into this hole in a press fit relationship. Various configurations on the shank of the implant, such as annular rings, can hold the implant in place. The toe is placed back into position and the tissue suture is closed.

In some embodiments of the present invention, a long midline medial incision is made commencing at mid proximal phalangeal shaft to distal one-third of the metatarsal shaft. The incision can be deepened by sharp and blunt dissection exposing the underlying capsule. The subcutaneous tissue can be dissected free from the underlying capsule and the toe disarticulated to expose the end of the first phalanx bone. Three disc shaped sizing guides can be provided to help determine the correct implant diameter. In some embodiments, the correct implant diameter substantially matches the phalanges diameter. Choosing too large a size can result in drilling through the plantar cortex when making the cavity for the implant. After the decision implant diameter has been made, a wire, such as a 1.6 mm K-wire, is drilled axially through the cortical bone and into the intramedullary canal. The purpose of the wire is to guide one or more reamers as they create a cavity for the implant.

In some embodiments, the reamers can be provided in a set consisting of two reamers for each diameter implant. The first reamer used in the procedure can be a pilot reamer useful for drilling a pilot hole and squaring off the end of the phalanx. In some embodiments, the reamer is chucked in a surgical drill and placed over the K-wire projecting from the phalanx. After reaming the hole, any bone projecting above the flattened surface can be removed using a small bone rongeur. A second reamer useful for creating a tapered area of the cavity can be chucked in a drill and also passed over the K-wire. After reaming, the K-wire can be removed from the bone.

The metatarsal head requires smoothing and removal of marginal osteophytes from the lateral dorsal, and medial aspects of the metatarsal head that would interfere with the motion of the implant. A smoother is provided which has a textured concave surface that can be used to smooth the metatarsal by positioning in contact with the head and manipulating it back and forth. A curved osteotome or a small rongeur can also be used. Smoothness should be judged by palpation of the articulating surface of the metatarsal.

A set of trial devices can be used to determine which implant height will provide the correct amount of ligament tension in the joint. The trial can be chosen and inserted into the reamed hole. In some embodiments, the trials do not have stabilization means on the fixation shank, so press fitting into the bone is not required. The joint can be reduced and examined for tension and motion. If the reduced and neutrally positioned articulation cannot be separated with the application of modest manual traction on the toe, the trial can be removed and replaced with one having less projection. An overly tight joint can result in limited motion and contraction hallux deformity post surgery.

The choice of the implant diameter can be reassessed at this point, and if a larger diameter would be beneficial, the K-wire can be reinstalled and a larger size of reamers can be used to prepare the phalanges for the larger diameter implant.

Once the proper implant size has been chosen, the implant can be placed in an impacting device. This device can hold the implant while it is placed within the phalanges. The implant can be installed by plantarflexing the toe. In some embodiments, the end of the impacting device is struck with a mallet until the implant is bottomed out and the tapered part of the implant rests on the cortical bone in the reamed hole.

In some embodiments, the joint capsule is then approximated and sutured, preferably covering the implant completely. The superficial fascia and skin can then be approximated and sutured and a dry sterile semi-compression dressing applied. Post operative range of motion can then be established.

The biomaterial can be prepared from any suitable material. Generally, a material is suitable if it has appropriate biostability, biodurability and biocompatibility characteristics. Typically, the materials include polymeric materials, having an optimal combination of such properties as biostability, biodurability, biocompatibility, physical strength and durability, and compatibility with other components (and/or biomaterials) used in the assembly of a final composite.

Examples of polymeric materials that may be suitable in some applications, either alone or in combination, include polyurethane, available from Polymer Technology Group Incorporated under the names Bionate,™ Biospan,™ and Elasthane™, available from Dow Chemical Company under the name Pellethane,™ and available from Bayer Corp. under the names Bayflex,™ Texin,™ and Desmopan;™ ABS, available from GE Plastics under the name Cycolac™, and available from Dow Chemical Company under the name Magnum;™ SAN, available from Bayer Plastics under the name Lustran;™ Acetal, available from Dupont under the name Delrin,™ and available from Ticona GmbH and/or Ticona LLC (Ticona) under the name Celcon;™ polycarbonate, available from GE Plastics under the name Lexan,™ and available from Bayer Corp. under the name Makrolon;™ polyethylene, available from Huntsman LLC, and available from Ticona under the names GUR 1020™ and GUR 1050;™ polypropylenes, available from Solvay Engineered Polymers, Inc. under the name Dexflex;™ aromatic polyesters, available from Ticona; polyetherimide (PEI), and available from GE Plastics under the name Ultem;™ polyamide-imide (PAI), available from DSM E Products under the name Torlon;™ polyphenylene sulfide, available from Chevron Phillips Chemical Company LP under the name Ryton;™ polyester, available from Dupont under the name Dacron;™ polyester thermoset, available from Ashland Specialty Chemical Company under the name Aropol;™ polyureas; hydrogels, available from Hydromer Inc.; liquid crystal polymer, available from Ticona under the name Vectra;™ polysiloxanes, available from Nusil Technologies, Inc.; polyacrylates, available from Rohm & Haas under the name Plexiglas;™ epoxies, available from Ciba Specialty Chemicals; polyimides, available from Dupont under the names Kapton,™ and Vespel;™ polysulfones, available from BP Amoco Chemicals under the name Udel,™ and available from BASF Corporation under the name Ultrason;™ PEAK/PEEK, available from Victrex under the name Victrex PEAK;™ as well as biopolymers, such as collagen or collagen-based materials, chitosan and similar polysaccharides, and combinations thereof Of course, any of the materials suitable for use in a composite or single biomaterial implant may be structurally enhanced with fillers, fibers, meshes or other structurally enhancing means.

The present invention provides a biomaterial having an improved combination of properties for the preparation, storage, implantation and long term use of medical implants. The improved properties correspond well for the preparation and use of an implant having both weight bearing and/or articulating functions, and preferably in the form of an implant for interpositional arthroplasty.

In turn, a preferred biomaterial of this invention provides an optimal combination of properties relating to wear resistance, congruence, and cushioning while meeting or exceeding requirements for biocompatibility, all in a manner that serves to reduce the coefficient of friction at the major motion interface.

Wear resistance can be assessed by determining parameters such as DIN abrasion and flexural stress strain fatigue resistance. A preferred implant will have sufficient wear resistance to avoid the generation of clinically significant particulate debris over the course of the implant's use.

Congruence can be assessed by determining parameters such as tensile modulus compressive modulus, and hardness, to determine the manner and extent to which the implant will conform itself to possible other components of the implant itself and/or to bone or surrounding tissue.

Cushioning can be assessed by determining such parameters as hardness, compressive modulus, and tensile modulus, to determine the elastomeric nature of the material, and in turn, its suitability for use in a weight bearing joint. More elastomeric materials will generally provide greater comfort in weight bearing applications, particularly if the other physical properties can be maintained.

Applicant has discovered that improved wear resistance, congruence, and/or cushioning toughness can be achieved without undue effect on other desired properties, such as abrasion, hardness, specific gravity, tear resistance, tensile strength, ultimate elongation, and biocompatibility. Moreover, Applicant has discovered that such properties can themselves be provided in varying forms, as between first and second biomaterials of a composite of the present invention.

A polymeric biomaterial of this invention can be prepared using any suitable means, including by curing the polymer ex vivo. The composition can be used in any suitable combination with other materials, including other compositions of the same or similar nature, as well as other materials such as natural or synthetic polymers, metals, ceramics, and the like.

The invention further provides a method of preparing the composition, a method of using the composition, implants that comprise the composition, as well as methods of preparing and using such implants.

The biomaterial used in this invention preferably includes polyurethane components that are reacted ex vivo to form a polyurethane (“PU”). The formed PU, in turn, includes both hard and soft segments. The hard segments are typically comprised of stiffer oligourethane units formed from diisocyanate and chain extender, while the soft segments are typically comprised of one or more flexible polyol units. These two types of segments will generally phase separate to form hard and soft segment domains, since they tend to be incompatible with one another. Those skilled in the relevant art, given the present teaching, will appreciate the manner in which the relative amounts of the hard and soft segments in the formed polyurethane, as well as the degree of phase segregation, can have a significant impact on the final physical and mechanical properties of the polymer. Those skilled in the art will, in turn, appreciate the manner in which such polymer compositions can be manipulated to produce cured and curing polymers with desired combination of properties within the scope of this invention.

The hard segments of the polymer can be formed by a reaction between the diisocyanate or multifunctional isocyanate and chain extender. Some examples of suitable isocyanates for preparation of the hard segment of this invention include aromatic diisocyanates and their polymeric form or mixtures of isomers or combinations thereof, such as toluene diisocyanates, naphthalene diisocyanates, phenylene diisocyanates, xylylene diisocyanates, and diphenylmethane diisocyanates, and other aromatic polyisocyanates known in the art. Other examples of suitable polyisocyanates for preparation of the hard segment of this invention include aliphatic and cycloaliphatic isocyanates and their polymers or mixtures or combinations thereof, such as cyclohexane diisocyanates, cyclohexyl-bis methylene diisocyanates, isophorone diisocyanates and hexamethylene diisocyanates and other aliphatic polyisocyanates. Combinations of aromatic and aliphatic or arylakyl diisocyanates can also be used.

The isocyanate component can be provided in any suitable form, examples of which include 2,4′-diphenylmethane diisocyanate, 4,4′-diphenylmethane diisocyanate, and mixtures or combinations of these isomers, optionally together with small quantities of 2,2′-diphenylmethane diisocyanate (typical of commercially available diphenylmethane diisocyanates). Other examples include aromatic polyisocyanates and their mixtures or combinations, such as are derived from phosgenation of the condensation product of aniline and formaldehyde. It is suitable to use an isocyanate that has low volatility, such as diphenylmethane diisocyanate, rather than more volatile materials such as toluene diisocyanate. An example of a particularly suitable isocyanate component is the 4,4′-diphenylmethane diisocyanate (“MDI”). Alternatively, it can be provided in liquid form as a combination of 2,2′-, 2,4′- and 4,4′-isomers of MDI. In a preferred embodiment, the isocyanate is MDI and even more preferably 4,4′-diphenylmethane diisocyanate.

In one embodiment of the invention, the isocyanate is 4,4′-diphenylmethane diisocyanate (as available from Bayer under the tradename Mondur M), from preferably about 20 to 60 weight percent, more preferably from about 30 to 50 weight percent. The actual amount of isocyanate used should be considered in combination with other ingredients and processing parameters, particularly including the amount of chain extender (such as butanediol (BDO)) used, since the combination typically determines the hard segment component, and in turn hardness, of the corresponding cured polymer. Hardness correlates in a generally proportional fashion with the combined weights of MDI and BDO, such that compositions having between 30 and 60 total weight percent (MDI+BDO) are generally useful, with those compositions having between about 50 to about 60 total weight percent being somewhat harder, and particularly useful for either the first (femoral contacting) biomaterial and surface of a composite implant or for implants having a single biomaterial providing both first and second surfaces. By contrast, compositions having between about 40 to about 50 total weight percent are somewhat more congruent and cushioning, though less wear resistant, and therefore are preferred for use as the second biomaterial, e.g., tibial contacting surface, of a composite implant as described herein.

Some examples of chain extenders for preparation of the hard segment of this invention include, but are not limited, to short chain diols or triols and their mixtures or combinations thereof, such as 1,4-butane diol, 2-methyl-1,3-propane diol, 1,3-propane-diol ethylene glycol, diethylene glycol, glycerol, tri-methylpropane, cyclohexane dimethanol, triethanol amine, and methyldiethanol amine. Other examples of chain extenders for preparation of the hard segment of this invention include, but are not limited to, short chain diamines and their mixtures or combinations thereof, such as dianiline, toluene diamine, cyclohexyl diamine, and other short chain diamines known in the art.

The soft segment consists of urethane terminated polyol moieties, which are formed by a reaction between the polyisocyanate or diisocyanate or polymeric diisocyanate and polyol. Examples of suitable diisocyanates are denoted above. Some examples of polyols for preparation of the soft segment of this invention include but are not limited to polyalkylene oxide ethers derived form the condensation of alkylene oxides (e.g. ethylene oxide, propylene oxide, and blends thereof), as well as tetrahyrofuran based polytetramethylene ether glycols, polycaprolactone diols, polycarbonate diols and polyester diols and combinations thereof. In a preferred embodiment, the polyols are polytetrahydrofuran polyols (“PTHF”), also known as polytetramethylene oxide (“PTMO”) or polytetramethylene ether glycols (“PTMEG”). Even more preferably, the use of two or more of PTMO diols with different molecular weights selected from the commercially available group consisting of 250, 650, 1000, 1400, 1800, 2000 and 2900.

Two or more PTMO diols of different molecular weight can be used as a blend or separately, and in an independent fashion as between the different parts of a two part system. The solidification temperature(s) of PTMO diols is generally proportional to their molecular weights. The compatibility of the PTMO diols with such chain extenders as 1,4-butanediol is generally in the reverse proportion to the molecular weight of the diol(s). Therefore the incorporation of the low molecular weight PTMO diols in a “curative” (part B) component of a two part system, and higher molecular weight PTMO diols in the prepolymer (part A) component, can provide a two-part system that can be used at relatively low temperature. In turn, good compatibility of the low molecular weight PTMO diols with such chain extenders as 1,4-butanediol permits the preparation of two part systems with higher prepolymer to curative) volume ratio. Amine terminated polyethers and/or polycarbonate-based diols can also be used for building of the soft segment.

In one embodiment of the invention, the polyol is polytetramethyleneetherglycol 1000 (as available from E.I. du Pont de Nemours and Co. under the tradename Terathane 1000), preferably from about 0 to 40 weight percent, more preferably from about 10 to 30 weight percent, and perhaps even more preferably from about 22 to 24 weight percent, based on the total weight of the polymer. The polyol disclosed above may be used in combination with polytetramethyleneetherglycol 2000 (as available from E.I. du Pont de Nemours and Co. under the tradename Terathane 2000), preferably from about 0 to 40 weight percent, more preferably from about 10 to 30 weight percent, and perhaps even more preferably from about 17 to 18 weight percent, based on the total weight of the polymer.

In one embodiment, the biomaterial may include a chain extender. For example, the chain extender may be 1,4-butanediol (as available from Sigma Aldrich Corp.), preferably from about 1 to 20 weight percent, more preferably from 5 to 15 weight percent, to perhaps even more preferably from 12 to 13 weight percent, based on the total weight of the polymer.

The polyurethane can be chemically crosslinked, e.g., by the addition of multifunctional or branched OH-terminated crosslinking agents or chain extenders, or multifunctional isocyanates. Some examples of suitable crosslinking agents include, but are not limited to, trimethylol propane (“TMP”), glycerol, hydroxyl terminated polybutadienes, hydroxyl terminated polybutadienes (HOPB), trimer alcohols, Castor oil polyethyleneoxide (PEO), polypropyleneoxide (PPO) and PEO-PPO triols. In a preferred embodiment, HOPB is used as the crosslinking agent.

This chemical crosslinking augments the physical or “virtual” crosslinking of the polymer by hard segment domains that are in the glassy state at the temperature of the application. The optimal level of chemical cross-linking improves the compression set of the material, reduces the amount of the extractable components, and improves the biodurability of the PU. This can be particularly useful in relatively soft polyurethanes, such as those suitable for the repair of damaged cartilage. Reinforcement by virtual cross-links alone may not generate sufficient strength for in vivo performance in certain applications. Additional cross-linking from the soft segment, potentially generated by the use of higher functional polyols can be used to provide stiffer and less elastomeric materials. In this manner a balancing of hard and soft segments, and their relative contributions to overall properties can be achieved.

In one embodiment, the chemical cross-linking agent is 2-ethyl-2-(hydroxymethyl)-1,3-propanediol (also known as trimethylolpropane, as available from Sigma Aldrich Corp.), preferably from about 0 to 5 weight percent, more preferably from about 0.1 to 1 weight percent, and perhaps even more preferably from about 0.15 to 0.3 weight percent, based on the total weight of the polymer.

Additionally, and optionally, a polymer system of the present invention may contain at least one or more biocompatible catalysts that can assist in controlling the curing process, including the following periods: (1) the cure induction period, and (2) the full curing period of the biomaterial. Together these two periods, including their absolute and relative lengths, and the rate of acceleration or cure within each period, determine the cure kinetics or profile for the composition. In some embodiments, however, a catalyst is not included. For instance embodiments in which the biomaterial is heated in the course of curing, such as in a heated mold in the manner described herein, can performed without the use of a catalyst.

Some examples of suitable catalysts for preparation of the formed PU of this invention include, but are not limited to, tin and tertiary amine compounds or combinations thereof such as dibutyl tin dilaurate (DBTDL), and tin or mixed tin catalysts including those available under the tradenames “Cotin 222”, “Fomrez UL-22” (Crompton Corp.), “dabco” (a triethylene diamine from Sigma-Aldrich), stannous octanoate, trimethyl amine, and triethyl amine.

In one embodiment of the invention, the catalyst is bis-(dodecylthio)-dimethylstannane (available from Crompton Corp. as Fomrez catalyst UL-22), preferably from about 0 to 2 weight percent, more preferably from about 0 to 1 weight percent, and perhaps most preferably from 0.0009 to 0.002 weight percent, based on the total weight of the polymer.

Further, a polymer stabilizer additive useful for protecting the polymer from oxidation may be included. In one embodiment of the invention, the additive is pentaerythritol tetrakis (3-(3,5-di-tert-buyl-4-hydroxyphenyl)proprionate (available from Ciba Specialty Chemicals, Inc. as Irganox 1010), preferably from about 0 to 5 weight percent, more preferably about 0.1 to 1 weight percent, and perhaps even more preferably about 0.35 to 0.5 weight percent, based on the total weight of the polymer.

Optionally, other ingredients or additives can be included, for instance, a reactive polymer additive can be included from the group consisting of hydroxyl- or amine-terminated compounds selected from the group consisting of poybutadiene, polyisoprene, polyisobutylene, silicones, polyethylene-propylenediene, copolymers of butadiene with acryolnitrile, copolymers of butadiene with styrene, copolymers of isoprene with acrylonitrile, copolymers of isoprene with styrene, and mixtures of the above. Other additives may also be optionally provided. For example, catalysts such as Dabco, antioxidants such as vitamin E, hydrophobic additives such as hydroxyl-terminated polybutadiene, and dye green GLS, singularly or in combination, may be included in the polymer formulation.

Suitable compositions for use in the present invention are those polymeric materials that provide an optimal combination of properties relating to their manufacture, application, and in vivo use. In the uncured state, such properties include component miscibility or compatibility, processability, and the ability to be adequately sterilized or aseptically processed and stored. While the composition is curing, suitable materials exhibit an optimal combination of cure kinetics and exotherm. In the cured state, suitable compositions exhibit an optimal combination of such properties as abrasion, hardness, specific gravity, tear resistance, tensile strength, ultimate elongation, and biocompatibility.

The composition of the present invention provides a polyurethane that can be prepared ex vivo. Particularly when formed ex vivo, products incorporating the composition of this invention may be made in advance of their use, on a commercial scale, and under stringent conditions.

Polymeric biomaterials of this invention, including preferred polyurethanes can be prepared using automated manufacturing processes within the skill of those in the art. A preferred manufacturing method, for instance, includes the use of multichannel dispensing equipment to inject the polymer. Such equipment is well suited to high precision applications, having a variable or fixed number of channels, some have all channels dispensing the same volume while in others the volume can be set by channel, some have all channels dispensing the same fluid, while others allow for different fluids in different channels. The dispensing can be automated repetitive or manual. Suitable devices for metering, mixing and dispensing materials such as urethanes are commercially available from a variety of sources, including for instance from Adhesive Systems Technology Corp., 9000 Science Center Drive, New Hope, Minn. 55428.

Furthermore, polymeric biomaterials of this invention may be cured in a heated mold. The mold may receive the contents of the polymeric biomaterial before it is cured. In one embodiment, a permanent enclosed mold is used to form at least a part of the implant. Such a mold may be similar to a standard injection mold and have the ability to withstand large clamping forces. Further, such a mold may include runners and/or vents to allow material to enter and air to exit. Such a mold may be constructed from metals, polymers, ceramics, and/or other suitable materials. The mold may be capable of applying and controlling heat to the biomaterial to accelerate curing time. In some embodiments, the mold may be coated with a release coating agent to facilitate ease of removal of the cured biomaterial from the mold. Examples of suitable release agents include Teflon,™ silicone, florinated ethylene propylene (FEP), dichronite, gold, and nickel-Teflon combinations, various types of which are commercially available from a variety of sources, e.g., McLube Division of McGee Industries. In addition, the mold may be provided in two separable parts to further facilitate removal of the cured biomaterial.

Further, time and temperature parameters can be modified in processing to change the characteristics of the implant. A time temperature profile may be selected to achieve certain implant properties. In embodiments formed with a heated mold as described above, those skilled in the art will appreciate the manner in which both the temperature of the mold as well as the time biomaterial is maintained can be adjusted to change the characteristics of the molded implant.

In the embodiment in which an ex vivo curing polymer is used, the present invention preferably provides a biomaterial in the form of a curable polyurethane composition comprising a plurality of parts capable of being at least partially mixed at a time before use, the parts including: (1) a polymer component comprising the reaction product of one or more polyols, and one or more diisocyanates, and (2) a curative component comprising one or more chain extenders, one or more catalysts, and optionally, one or more polyols and/or other optional ingredients.

In some embodiments, long term congruence of the biomaterial is facilitated by its hydration in vivo, permitting the biomaterial to become more pliable, and in turn, facilitate congruence with the tibial plateau. In turn, an increase in hydration and/or changes in temperature can improve the fit and mechanical lock between the implant and the tibial plateau. The biomaterial may be hydrated ex vivo and/or in vivo, both before and after the composition is cured. Preferably, the biomaterial may be further hydrated within the joint site after the composition in order to enhance both conformance and performance of the implant.

Implantable compositions of this invention demonstrate an optimal combination of properties, particularly in terms of their physical/mechanical properties, and biocompatibility. Such performance can be evaluated using procedures commonly accepted for the evaluation of natural tissue, as well as the evaluation of materials and polymers in general. In particular, a preferred composition, in its cured form, exhibits physical and mechanical properties that approximate or exceed those of the natural tissue it is intended to provide or replace. Fully cured polymeric (e.g., polyurethane) biomaterials within the scope of this invention provide an optimal combination of such properties as abrasion, compressive hardness, compressive modulus hardness, specific gravity, tear resistance, tensile strength, ultimate elongation, tensile modulus, and biocompatibility.

Physical/Mechanical Properties and Test Methods

Various properties of the composition of this invention can be evaluated for use in quality control, for predicting service performance, to generate design data, to determine compliance with established standards, and on occasion, to investigate failures. See, for instance, Handbook of Polymer Testing: Physical Methods, edited by Roger Brown, Marcel Dekker, Inc., New York, N.Y. (1999), the disclosure of which is incorporated herein by reference. Suitable properties include those dealing with a) mass, density and dimensions, b) processability, c) strength and stiffness (including compressive hardness, compressive modulus, tensile stress-strain, flexural stress-strain, flexibility, and tear tests), c) fatigue and wear (including abrasion resistance and hardness), d) time dependent properties (such as creep, stress relaxation, compression set, tension set), e) effect of temperature (such as thermal expansion, shrinkage, and thermal oxidative aging), f) environmental resistance, and g) and biocompatibility parameters.

Of particular note are those properties that lend themselves to the preparation, delivery and long term use of improved implants having an articulating surface, and preferably for long term weight bearing use.

The preferred property ranges given below are only relevant to certain embodiments of the invention. It will be appreciated by those reasonably skilled in the art that materials having one or more properties outside the scope of the preferred ranges given below are suitable for use with the present invention.

Abrasion values for a polymer can be determined with a rotating cylindrical drum device, known as a DIN abrader. A loaded cylindrical test piece is traversed along an abrasive cloth attached to a rotating drum, and the mass loss is measured after a specified length of travel. Advantages of this device include the use of a test piece small enough to be cut from a product or a comparatively thin sheet and a much reduced risk of abrasive contamination caused by debris or smearing. The result can be expressed with the abrasion resistance index, which is the ratio of the volume loss of a black standard rubber sample to the volume loss of the test sample.

The polymer preferably provides a DIN abrasion value of less than about 70 mm³, more preferably less than about 60 mm³ and most preferably less than about 50 mm³, as determined by ASTM Test Method D5963-96 (“Standard Test Method for Rubber Property Abrasion Resistance Rotary Drum Abrader”). DIN abrasion values of greater than about 70 mm³ tend to exhibit wear rates that are too great for longer term use as articulating surface.

Biomaterial can be formed into standardized (e.g., puck-like) implant shapes and subjected to conditions intended to replicate, while also meet and exceed physiological conditions. Preferred biomaterials of this invention are able to withstand one million cycles (approximately equivalent to 1 year implantation), and more preferably greater than 5 million cycles (approximately equivalent to 5 years) before generating unsuitable debris.

Flexural stress/strain fatigue can be measured in a variety of ways. Using the standardized shape as described above, samples can be compressively loaded in cycles of increasing loads, and the stress strain fatigue can be plotted verses the number of cycles.

As another example, flexural stress/strain fatigue can be determined by a three point bending test, in which a standardized implant sample shape is supported at its anterior and posterior ends. A cyclical load is applied to the sample in an area substantially between the two supports to provide a deflection of approximately 4 mm, and the total number of cycles until failure is recorded.

Biomaterials formed into implant shapes in accordance with the present invention, under conditions intended to meet and exceed physiological conditions, are preferably able to withstand one million cycles (approximately equivalent to 1 year implantation), and more preferably greater than five million cycles (approximately equivalent to 5 years implantation) in a test similar to the one described above.

Fracture toughness can generally be determined by a number of methods. For example, fracture toughness can be measured by tests similar to ASTM Test Method D5045-99.

Preferably, the polymer provides a peak load fracture toughness of at least about 50 lbs, more preferably more than about 80 lbs, and most preferably more than about 110 lbs. Further, the polymer preferably provides an energy to break fracture toughness of greater than about 15 lb-in, more preferably greater than about 25 lb-in, and most preferably greater than about 30 lb-in. These values may be obtained with tests similar to ASTM Test Method D5045-99.

The term hardness has been applied to scratch resistance and to rebound resilience, but for polymers it is taken to refer to a measure of resistance to indentation. The mode of deformation under an indentor is a mixture of tension, shear, and compression. The indenting force is usually applied in one of the following ways: Application of a constant force, the resultant indentation being measured, measurement of the force required to produce a constant indentation, or use of a spring resulting in variation of the indenting force with depth of indentation.

A biomaterial of this invention preferably provides a hardness value when hydrated of less than about 75 Shore D, more preferably less than about 70 Shore D, and most preferably less than about 60 Shore D, as determined by ASTM Test Method D2240. In some embodiments, hydration of the biomaterial may lower the shore hardness value.

In one method of determining specific gravity, a test piece is provided weighing a minimum of 2.5 grams, which can be of any shape as long as the surfaces are smooth and there are no crevices to trap air. The test piece is weighed in air and then in water using a balance accurate to 1 mg. The test piece can be suspended by means of a very fine filament, the weight of which can be included in the zero adjustment of the balance and its volume in water ignored. The specific gravity is calculated from the difference in measurements.

The polymer preferably provides a specific gravity of about 1 to 2 g/cm³, more preferably about 1 to 1.5 g/cm³, and most preferably about 1.15 to 1.17 g/cm³, as determined by ASTM Test Method D792.

A tear test may be used to measure tear strength. In a tear test, the force is not applied evenly but is concentrated on a deliberate flaw or sharp discontinuity in the sample and the force to produce continuously new surface is measured. This force to start or maintain tearing will depend in a complex manner on the geometry of the test piece and the nature of the discontinuity.

Preferably, a biomaterial of this invention provides a tear strength value in the Die C configuration of greater than about 400 pounds per linear inch (PLI), more preferably greater than about 600 PLI, and most preferably greater than about 800 PLI, and a value in the Die T configuration of preferably greater than about 100 PLI, more preferably greater than about 150 PLI, and most preferably greater than about 250 PLI, as determined by ASTM Test Method D624.

To measure tensile modulus, tensile strength, and ultimate elongation, a test piece of the material is stretched until it breaks, and the force and elongation at various stages is measured. A tensile machine is used to perform this test. Generally, the basic elements of a tensile machine are grips to hold the test piece, a means of applying a strain (or stress), a force-measuring instrument, and an extensometer.

The polymer preferably provides a tensile modulus at 100% elongation value of about 1,000 to 10,000 psi, more preferably about 2,000 to 5,000 psi, and most preferably about 2,500 to 4,500 psi, as determined by ASTM Test method D412.

The polymer preferably provides a tensile modulus at 200% elongation value of about 1,000 to 10,000 psi, more preferably about 2,000 to 6,000 psi, and most preferably about 2,500 to 5,000 psi, as determined by ASTM Test method D412.

The polymer preferably provides a tensile strength value of greater than about 6,000 psi, more preferably greater than about 6,500 psi, and most preferably greater than about 7,000 psi., as determined by ASTM Test Method D412.

Preferably, the polymer provides an ultimate elongation of greater than about 200%, more preferably greater than about 250%, and most preferably greater than about 300%, as determined by ASTM Test Method D412.

To measure compressive modulus and compressive strength, a sample is again formed in a standardized (e.g., puck) shape and varying compressive loads are applied to the sample in order to develop a corresponding curve. The compressive modulus can be determined from this curve. Compressive strength may be determined by applying increasing loads to a sample until the sample fails.

Preferably, the sample implant provides an compressive modulus of greater than about 4,000 psi, more preferably greater than about 4,500 psi, and most preferably greater than about 5,000 psi, as determined in the manner described above.

Preferably, the sample implant also provides a compressive strength of greater than about 6,000 psi, more preferably greater than about 7,000 psi, and most preferably greater than about 8,000 psi, as determined by a test similar to the one described above.

Water absorption may be determined in a variety of ways. A suitable method for measuring water absorption is to submerge a sample of the test material, with an implant-type geometry, in a saline solution. Once the sample and saline solution reach equilibrium at 37 degrees Celsius, which may take a month or longer, the sample is removed and weighed to determine its water absorption.

Preferably, the polymer provides a water absorption value less than about 5% at 37 C, more preferably less than about 3% at 37 C, and most preferably less than about 2% at 37 C, as determined by a test similar to the one described above.

The medical-grade polyurethane resins were evaluated for biocompatibility in accordance with ISO 10993: Biological Evaluation of Medical Devices and FDA G95-1: Required Biocompatibility Training and Toxicology Profiles for Evaluation of Medical Devices. The biological effects of the resin, such as cytotoxicity, sensitization, genotoxicity, implantation, chronic toxicity, and carcinogenicity, were studied. The tests were conducted in accordance with the FDA Good Laboratory Practice (GLP) Regulation.

The following tests were conducted to determine if the polymer is biocompatible: 1) ISO MEM elution using L-929 mouse fibroblast cells; 2) ISO agarose overlay using L-929 mouse fibroblast cells; 3) ISO acute systemic injection test; 4) ISO intracutaneous reactivity test; 5) ISO guinea pig maximization sensitization test; 6) Material mediated rabbit pyrogen test; 7) In vitro genotoxicology test; and 8) ISO muscle implantation study in the rabbit with histology-1 week. The results of the eight selected screening biocompatibility tests above show that the polymer passes all the tests and is considered biocompatible.

In an alternative embodiment, the implant can be provided by any of a series of metals, including titanium, stainless steel, cobalt chrome millithium alloys and tantalum. Other surface materials can include various ceramics and biologic polymers.

Numerous characteristics and advantages of the invention covered by this document have been set forth in the foregoing description. It will be understood, however, that this disclosure is, in many respects, only illustrative. Changes can be made in details, particularly in matters of shape, size and ordering of steps without exceeding the scope of the invention. The scope of this invention is, of course, defined in the language in which the appended claims are expressed.

What is claimed is:

1. A system for the creation or modification of an othropedic joint within a mammalian body, the system comprising a polymeric implant that provides a major surface adapted to be positioned against a metatarsal bone and an end adapted to be retained within a phalange.

2. An implant in accordance with claim 1 wherein the implant comprises a body and a shank.

3. An implant in accordance with claim 2 wherein the implant includes a means for stabilization.

4. An implant in accordance with claim 3 wherein the means for stabilization include barbs.

5. An implant in accordance with claim 3 wherein the means for stabilization include sharp points.

6. An implant in accordance with claim 3 wherein the means for stabilization include splines.

7. An implant in accordance with claim 3 wherein the means for stabilization include diamond patterns.

8. An implant in accordance with claim 1 wherein the implant comprises a biomaterial.

9. An implant in accordance with claim 8 wherein the biomaterial comprises a polyurethane.

10. An implant according to claim 9 wherein the polyurethane is biocompatible with respect to cytotoxicity, sensitization, genotoxicity, chronic toxicity, and carcinogenicity.

11. An implant according to claim 9 wherein polyurethane has a Shore hardness of at least about 60 D or less.

12. A kit for a positional arthroplasty system, the kit comprising:
a) an implant that provides a major surface adapted to be positioned against a metatarsal bone and an end adapted to be retained within a phalange, and
b) one or more devices adapted to perform one or more steps selected from the group consisting of preparing the joint to receive an implant, determining an appropriate implant size for a particular joint, inserting the implant into the joint, and/or securing the implant to a desired extent.

13. A kit according to claim 12 wherein the kit includes a includes an impactor.

14. A kit according to claim 12 wherein the kit includes a reamer.

15. A kit according to claim 12 wherein the kit includes a depth stop.

16. A kit according to claim 12 wherein the kit includes a bone smoother.

17. A kit according to claim 16 wherein the smoother is fenestrated.

18. A kit according to claim 12 wherein the kit includes a diameter gauge.

19. A method of repairing a metatarsophalangeal joint, comprising the steps of providing and implanting according to claim 1.

20. A metatarsophalangeal joint that includes an implant according to claim 1.

21. A kit comprising a tool useful for preparing a joint to receive an implant, an apparatus useful for determining an appropriate implant size for the joint, an apparatus useful for determining an appropriate implant thickness, and a tool useful for inserting the implant into the joint and/or securing the implant to a desired extent.

22. A device for implantation into a toe joint space within the body of a mammal, the device comprising a composite or monolith structure fabricated from a biocompatible, biodurable material that is adapted to be inserted into the joint compartment.

23. A device according to claim 22 wherein the implanted device is substantially free of anchoring portions that need to be attached to the bone, cartilage, ligaments or other tissue, yet by its design is capable of being used with minimal translation, rotation, or other undesired movement or dislocation within or from the joint space.

24. A device according to claim 23 wherein stability of the device within the joint space is provided by the fixation/congruency of the device to the one or the other of the two joint members.
